(19) United States
(12) Patent Application Publication
(10) Pub. No.: US 2015/0141349 A1
(43) Pub. Date: May 21, 2015

(54) Method and Composition for Alleviating Tumor Symptoms

(71) Applicants: Angiogene Pharmaceuticals Ltd., Watington, OX (GB); OXIGENE, Inc., south San Francisco, CA (US)

(72) Inventors: Peter David Davis, Watlington (GB); David J. Chaplin, Watlington (GB)

(73) Assignee: Angiogene Pharmaceuticals Ltd., Watlington (GB)

(21) Appl. No.: 14/406,725

(22) PCT Filed: Jun. 21, 2013
(86) PCT No.: PCT/US13/47016

§ 371 Date: Dec. 9, 2014

Related U.S. Application Data
(60) Provisional application No. 61/662,635, filed on Jun. 21, 2012.

(51) Int. Cl. A61K 31/661; A61K 38/12

(52) U.S. Cl. CPC A61K 31/661; A61K 38/12
USPC 514/21.1; 514/130

(57) ABSTRACT

This invention relates to methods and compositions for treating carcinoid syndrome and other adverse symptoms associated with tumor-producing neuroendocrine tumors, said methods comprising administering a therapeutically effective amount of a vascular disrupting agent, or a pharmaceutically acceptable salt thereof, to a subject having a hormone producing neuroendocrine tumor. In preferred implementations, the vascular disrupting agent is combretastatin A-4 phosphate, combretastatin A-1 diphosphate, or a pharmaceutical acceptable salt thereof.

### I. FIELD

This invention relates to the treatment of mammals having a tumor producing one or more hormones, particularly carcinoid tumors.

### II. INTRODUCTION

Cancer is a leading cause of death in the industrialized world and despite years of research, many types of cancer lack an effective therapeutic treatment. According to the American Cancer Society, there are approximately 5,000 new carcinoid tumors diagnosed in the US each year. An additional 3,000 neuroendocrine tumors (NETs) are diagnosed annually.

Assuming similar incidence rates, this translates to 16,000 new cases of carcinoid and NETs annually in the combined markets of EU/JP. Although the underlying cause is unclear, there is a strong consensus that the incidence of carcinoid tumors is increasing. Age-adjusted incidence grew 6.3% per annum between 1973 and 1997 (Maggard, 2002 Ann. Surgery 240:117-22) and more recent studies have suggested that the trend has continued (Yao, 2008 J Clin Oncol 26:3063-3072). In terms of tumor sites the increase in carcinoid has been primarily in sites including the small intestine, (the most common site and cause of the great majority of carcinoid syndrome cases) rectum and stomach while carcinoid of the appendix (which does not usually give rise to carcinoid syndrome) has remained constant.

A variety of benign or cancerous neuroendocrine tumors produce hormones that severely impact on the patient's quality of life. The released hormones can act locally, producing local symptoms such as pain, or remotely, giving rise to a variety of metabolic disorders. For example neuroendocrine tumors, including carcinoids and pancreatic neuroendocrine tumors, can generate hormones such as serotonin, chromogranin, neurotensin, vasoactive intestinal peptide, histamine, dopamine, kallikrein, substance P, insulin, prostaglandin, glucagon, gastrin, adrenocorticotropic hormone (ACTH), somatostatin, and parathyroid hormone.

Release of such hormones can result in raised local or circulating levels of such substances and consequently to debilitating symptoms. For example serotonin release, alone or with other hormones, in metastatic carcinoid patients can result in diarrhea and flushing. Additional symptoms may include abdominal pain, heart disease, wheezing, bloating, and sweating. Insulinomas secrete insulin leading to spontaneous or fasting hypoglycemia and neuroglycopenic symptoms. Gastrinomas produce gastrin, leading to marked gastric acid hypersecretion leading to peptic ulcers, diarrhea and gastroesophageal reflux disease. Vasoactive intestinal peptide secreted by VIPomas leads to severe watery diarrhea and glucagon from glucagonomas can lead to necrolytic migratory erythema, diabetes, weight loss, anemia, hypoaminoacidosis and venous thrombosis. Pituitary adenomas can secrete prolactin or growth hormone along with other hormones such as insulin-like growth factor 1 (IGF-1). Growth hormone secreted by pituitary tumors can give rise to acromegaly. Increased tumor-derived prolactin can produce hypogonadism, infertility and hyposexuality, among other symptoms.

Patients with a carcinoid primary, particularly mid-gut primary, that has metastasized to the liver have raised levels of tumour-derived hormones which impact not only the morbidity but also survival. Many patients develop overt and debilitating symptoms from the hormone release—carcinoid syndrome. Longer-term consequences of such hormone release are life threatening. Carcinoid tumours are resistant to chemotherapy and the only effective treatment of the symptoms of carcinoid syndrome is chronic administration of somatostatin analogs, such as octreotide, which, while having little impact on tumour growth, reduce the hormonal burden and relieve symptoms (the latter is in fact the clinical endpoint on which these agents are registered). Unfortunately only 50-75% of patients respond to somatostatin analog therapy and those that do typically start to lose responsiveness after around one year of treatment. Patients failing the therapy have few further treatment options.

The majority of conventional antitumor agents are ineffective against raised hormone levels arising from neuroendocrine and pituitary tumors. Limited success had been reported recently with agents that target Vascular Endothelial Growth Factor (VEGF). Although there are reports of these agents reducing tumor growth, it is clear that there is not a rapid and dramatic effect on hormone release. For example, after a prolonged treatment with the anti-VEGF antibody bevacizumab in combination with octreotide only 19% of neuroendocrine tumor patients achieved a meaningful reduction in urinary 5-HIAA (a marker for serotonin) and only 6% achieved a reduction in plasma levels of chromogranin A (Yao et al. J Clin Oncol 2008; 26:1316-23). These modest reductions are well within that which would be expected if the octreotide had been given alone and point to a lack of activity of the anti-VEGF antibody in this regard. There are also reports of activity, including partial radiological responses and, after significant periods of daily dosing, some reductions in hormone levels, against neuroendocrine tumors using tyrosine kinase inhibitors such as sunitinib, sorafenib and valatinib, and using mTOR inhibitors, such as everolimus. These agents inhibit a variety of targets and it is not clear which of the activities leads to the observed effect. The effects also come with a significant burden of toxicity.

There is, therefore, a need for agents that rapidly and effectively reduce hormone release from tumors, particularly neuroendocrine or pituitary tumors.

### III. SUMMARY OF THE INVENTION

One aspect of the invention provides methods of treating carcinoid syndrome. Preferably, the vascular disrupting agent is combretastatin A4 (CA4) or combretastatin A-4 phosphate (CA4P), or a pharmaceutically acceptable salt thereof. Alternatively, the vascular disrupting agent is combretastatin A1 (CA1) or combretastatin A-1 diphosphate (CA1dP), or a pharmaceutically acceptable salt thereof. The invention also provides compositions useful in the treatment of carcinoid syndrome and for use in the manufacture of a medicament for the treatment of carcinoid syndrome.

Another aspect of the invention provides methods of alleviating the symptoms associated with increased hormone production by a neuroendocrine tumor in a mammal, or compositions useful in the alleviating symptoms associated with having a tumor producing hormone or compositions useful in the manufacture of a medicament for alleviating symptoms associated with a tumor producing hormone. In particular it concerns methods for the reduction of hormone levels in a mammal suffering from a neuroendocrine tumor. In one aspect of the invention the method involves the administration to a mammal of a vascular disrupting agent, preferably combretastatin A-4 phosphate or combretastatin A-1 diphosphate. In another aspect of the invention the method involves the administration to a mammal of a somatostatin analog in combination with a vascular disrupting agent, preferably combretastatin A-4 phosphate or combretastatin A-1 diphosphate.

### IV. DETAILED DESCRIPTION

As used herein, a “therapeutically effective amount” of a vascular disrupting agent, or a therapeutically acceptable salt thereof, according to the present invention is intended to mean that amount of the vascular disrupting agent that will alleviate the adverse symptoms associated with increased hormone secretion by a neuroendocrine tumor, for example flushing and diarrhea.

As used herein, the term “treating” carcinoid syndrome, or other symptoms associated with a hormone-producing neuroendocrine tumor, is intended to mean inhibiting production of a tumor-generated hormone, decreasing levels of tumor-induced hormone and causing the regression and palliation of carcinoid syndrome, i.e., reducing the number of flushing or diarrheal events and/or increase quality of life. Other desired effects include, without limitation, decreases in abdominal pain, heart disease, wheezing, bloating, or sweating, and extending the survival of the subject beyond that which would otherwise be expected in the absence of such treatment.

As used herein, the terms “modulate”, “modulating” or “modulation” refer to changing the rate at which a particular process occurs, inhibiting a particular process, reversing a particular process, and/or preventing the initiation of a particular process. Accordingly, if the particular process is hormone production, the term “modulation” includes, without limitation, decreasing the rate at which hormone is produced; increasing the rate at which the hormone is degraded or cleared form the body; reversing adverse symptoms of high hormone levels (including flushing and diarrhea) and/or preventing development of such adverse symptoms, e.g. carcinoid heart disease.

As used herein, the term “prodrug” refers to a precursor form of the drug which is metabolically converted in vivo to produce the active drug. Thus, for example, combretastatin phosphate prodrug salts administered to an animal in accordance with the present invention undergo metabolic activation and regenerate combretastatin A-4 in vivo, e.g., following dissociation and exposure to endogenous non-specific phosphatases in the body, the drug which is metabolically converted in vivo to produce the active drug. Preferred prodrugs of the present invention include the phosphate, phosphoramidate, or amino acid acyl groups as defined herein. The phosphate ester salt moiety may also include (—OP(O)(O-alkyl)₂ or (—OP(O)(O⁻NH₄⁺)₂). In preferred embodiments, a prodrug of the invention comprises a substitution of a phenolic moiety or amine moiety of the active drug with a phosphate, phosphoramidate, or amino acid acyl group. A wide variety of methods for the preparation of prodrugs are known to those skilled in the art (see, for example, Pettit and Lippert, Anti-Cancer Drug Design, (2000), 15, 203-216).

“Neuroendocrine tumors” refers to a cell proliferative disorder arising from secretary cells of the endocrine and nervous system, and develop from different endocrine glands (such as the pituitary, the parathyroid or the neuroendocrine adrenal glands), from endocrine islets (for example in the pancreas) or from endocrine cells dispersed between exocrine cells throughout the digestive and respiratory tracts

A “hormone,” as used herein, refers to a naturally occurring substance, secreted by a cell, that transmits a signal from one cell to another, and thereby affects the metabolism or behavior of other cells possessing functional receptors for the hormone.

As used herein, the term “pharmaceutically acceptable salt” includes salts that are physiologically tolerated by a subject. Such salts are typically prepared from an inorganic and/or organic acid. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic (mesylate), ethanesulfonic, pantothenic, benzenesulfonic (besylate), stearic, sulfanilic, alginic, galacturonic, and the like. Other pharmaceutically acceptable salts include alkali metal cations such as Na, K, Li; alkali earth metal salts such as Mg or Ca; or organic amine salts such as those disclosed in PCT International Application Nos. WO02/22626 or WO00/48606 and U.S. Pat. Nos. 6,855,702 and 6,670,344, which are incorporated herein by reference in their entireties. Particularly preferred salts include organic amine salts such tromethamine (TRIS) and amino acid salts such as histidine. Other exemplary salts that can be synthesized using the methods of the invention include those described in U.S. Pat. No. 7,018,987, which is incorporated by reference herein.

Carcinoid syndrome is rare and is caused by carcinoid tumors—small, malignant or benign tumors that most commonly arise in the submucosa of the gastrointestinal tract. Carcinoid syndrome is the set of symptoms that may occur in patients who have carcinoid tumors. The syndrome occurs when carcinoid tumors overproduce substances, such as serotonin and chromogranin A, that normally circulate throughout the body. The serotonin produced by the carcinoid tumor is further metabolized to the most important serotonin metabolite, 5-hydroxyindoleacetic acid (5-HIAA).

This overproduction of serotonin and other hormones causes the symptoms of carcinoid syndrome which includes episodic flushing, diarrhea, wheezing, and potentially, the eventual development of carcinoid heart disease. Carcinoid tumors often do not produce noticeable symptoms until they spread to the liver. This is because most of the circulation from the gastrointestinal tract must pass through the liver before it reaches the rest of the body. The liver metabolizes most of the excess serotonin and other substances produced by the carcinoid tumors, preventing them from reaching tissues where they can cause symptoms. When carcinoid tumors metastasize to the liver, the substances they overproduce can more easily reach the bloodstream, and reach tissues where they can cause symptoms. Carcinoid patients may present with one or more of the individual symptoms of carcinoid syndrome. Certain implementations of the methods described herein are directed towards treatment of a mammal having one or more symptoms associated with carcinoid syndrome.

A variety of treatment options are available for carcinoid tumors and carcinoid syndrome, including surgical and medical therapies. Most patients require somatostatin analogs to help control the symptoms of carcinoid syndrome. Patients who no longer respond to somatostatin and other therapies with progression of disease and increasing symptoms have limited options including participation in a clinical trial. Measurement of 5-HIAA and chromogranin A can help determine the effectiveness of treatments for carcinoid tumors and carcinoid syndrome.

Vascular Disrupting Agents (VDAs) and are particularly useful in practicing the present invention. These are distinct in mechanism and effect to agents that inhibit angiogenesis (i.e., anti-angiogenic agents, such as bevacizumab). In contrast to anti-angiogenic agents, VDAs have a rapid effect on established tumor and other abnormal vasculature and therefore the overall effects of the two classes are different. Known VDAs include the colchinols (for example N-acetylcolchinol and colchinol prodrugs for example N-acetylcolchinol-O-phosphate), the combretastatins (for example combretastatin A1 and A4 and their prodrugs including combretastatin A4 phosphate, combretastatin A1 diphosphate, crolibulin and AVE8062). Other known reversible tubulin binding agents that can act as VDAs include denibulin, plinabulin, crinobulin and CYT997.

CA4P and CA1dP are reversible tubulin depolymerizing agents that target abnormal vascular endothelial cells causing them to round up, in the case of tumor vasculature exposure to such agent leads to selective tumor vascular shutdown, following by rapid and extensive tumor cell death. The predilection for tumor vasculature by CA4P and CA1dP rather than normal vasculature is due to the lack of smooth muscle or pericytes surrounding the tumor vasculature. The rapid and reversible tumor vascular shutdown leads to necrosis of the hypoxic central core of the tumor. Removal of this central core of a well vascularized tumor can significantly decrease the amount carcinoid syndrome-inducing hormone that is released into circulation within the subject's body.

Although vascular disrupting agents have been investigated as antitumor therapies there is no suggestion in the art that they would be effective in reducing hormone levels arising from neuroendocrine tumors or, in particular, effective in reducing the negative effects of carcinoid syndromes.

Carcinoid syndrome is debilitating and the options for treatment are quite limited. One option is hepatic artery embolization (HAE), which can partially cut off the blood supply to the tumour and usually results in rapid and dramatic lowering of hormones and improvement of symptoms (Strosberg et al 2006). Embolization with or without added chemotherapy (the chemo-containing regimens being known as HACE or TACE) can result in substantial shrinkage of carcinoid metastases but does not eliminate them; a recent review of available data shows a response rate of 55% to HAE in carcinoid (Yao 2005). This degree of activity compares favourably with that emerging from Phase II trials of newer agents, for example Avastin (18%), everolimus (13%), sorafenib (10%), imatinib (4%) sunitinib (2%), gefitinib (2%) and valatinib (0%). HAE is unique in its ability to shrink carcinoid metastases. The effectiveness of HAE relies on the tumour being fed largely from the arterial side of the hepatic circulation, which can be shut down without completely compromising the total supply to the liver, which derives the majority of its blood from the portal vein. Selective disruption of the blood supply to the tumour results in necrosis.

The HAE procedure is not without risk, largely due to induction of necrosis in the normal tissue of the liver and other organs. Selective embolization techniques are available but are inappropriate for multifocal disease. Moreover, repeat embolizations are less effective due to revascularisation from the portal supply. Thus although an initial embolization is often very successful in the control of hormones and symptoms, further treatments are much less effective. For example, in 84 patients with liver metastases from carcinoid or neuroendocrine pancreatic primaries, initial embolizations resulted in an 80% reduction in biochemical mediators (5-HIAA and chromogranin A) and a corresponding 80% reduction in symptoms (“clinical response”). However a second embolization series was much less effective with biochemical response rate of only 27% and a symptom response rate of 22% (Strosberg et al 2006). The toxicity and duration of HAE response thus does not support the routine use of HAE for treatment of metastatic carcinoid tumors and the resulting carcinoid syndrome.

Accordingly, one aspect of the present invention provides a method of treating carcinoid syndrome, the method comprising administering, to a mammal suffering from one or more symptoms of carcinoid syndrome, a therapeutically effective amount of a vascular disrupting agent. Preferably the vascular disrupting agent is a combretastatin. More preferably the vascular disruption agent is combretastatin A-4, a combretastatin A-4 prodrug (such as combretastatin A-4 phosphate), or a pharmaceutically acceptable salt thereof. Alternatively the vascular disrupting agent is combretastatin A1, a combretastatin A-1 prodrug (such as combretastatin A-1 diphosphate), or a pharmaceutically acceptable salt thereof.

Derived from the South African tree Combretum caffrum, combretastatins such as combretastatin A-4 (CA-4) were initially identified in the 1980's as a potent inhibitors of tubulin polymerization. CA-4, and other combretastatins (e.g. CA-1) have been shown to bind at or near the colchicine binding site on tubulin with high affinity. In vitro studies clearly demonstrated that combretastatins are potent cytotoxic agents against a diverse spectrum of tumor cell types in culture. CA4P and CA1dP, respective phosphate prodrugs of CA-4 and CA-1, were subsequently developed to combat problems with aqueous insolubility. A number of studies have shown that combretastatins cause extensive shut-down of blood flow within the tumor microvasculature, leading to secondary tumor cell death (Dark et al., Cancer Res., 57: 1829-34, (1997); Chaplin et al., Anticancer Res., 19: 189-96, (1999); Hill et al., Anticancer Res., 22(3):1453-8 (2002); Holwell et al., Anticancer Res., 22(2A):707-11, (2002). Blood flow to normal tissues is generally far less affected by CA4P and CA1dP than blood flow to tumors, although blood flow to some organs, such as spleen, skin, skeletal muscle and brain, can be transiently inhibited.

As used herein, the term “combretastatin” or “combretastatin compound” denotes at least one of the combretastatin family of compounds, derivatives or analogs thereof, their prodrugs (preferably phosphate prodrugs) and derivatives thereof, and salts of these compounds. Combretastatins include those anti-cancer compounds isolated from the South African tree Combretum caffrum, including without limitation, Combretastatins A-1, A-2, A-3, A-4, B-1, B-2, B-3, B-4, D-1, and D-2, and various prodrugs thereof, exemplified by Combretastatin A-4 phosphate (CA4P) compounds, Combretastatin A-1 diphosphate (CA1dP) compounds and salts thereof (see for example Pettit et al, Can. J. Chem., (1982); Pettit et al., J. Org. Chem., 1985; Pettit et al., J. Nat. Prod., 1987; Lin et al., Biochemistry, (1989); Pettit et al., J. Med. Chem., 1995; Pettit et al., Anticancer Drug Design, (2000); Pettit et al., Anticancer Drug Design, 16(4-5): 185-93 (2001)).

Combretastatin and combretastatin salts contemplated for use in the methods of the invention are described in WO 99/35150; WO 01/81355; WO02/022626; U.S. Pat. Nos. 4,996,237; 5,409,953; 5,561,122; 5,569,786; 6,538,038; 6,670,344; 6,855,702; 7,018,987; 7,078,552; and 7,279,466. Derivatives or analogs of combretastatins also are described in WO 06/138427; WO 036743; WO 05/007635, WO 03/040077, WO 03/035008, WO 02/50007, WO 02/14329; WO 01/12579, WO 01/09103, WO 01/81288, WO 01/84929, WO 00/48590, WO 00/73264, WO 00/06556, WO 00/35865, WO 99/34788, WO 99/48495, WO 92/16486, U.S. Pat. Nos. 7,125,906; 7,105,695; 7,105,501; 7,087,627; 7,030,123; 7,078,552; 7,030,123; 7,018,987; 6,992,106; 6,919,324; 6,846,192, 6,855,702; 6,849,656; 6,794,384; 6,787,672, 6,777,578, 6,723,858, 6,720,323, 6,433,012, 6,423,753, 6,201,001, 6,150,407, 6,169,104, 5,731,353, 5,674,906, 5,430,062, 5,525,632, 4,996,237 and 4,940,726.

The method of the invention can further comprise co-administering a second therapeutic agent, such as a somatostatin analog or chemotherapeutic agent, to the subject. “Co-administration”or “co-administering” can be in the form of a single formulation (combining, for example, CA4P and octreotide with pharmaceutically acceptable excipients, optionally segregating the two active ingredients in different excipient mixtures designed to independently control their respective release rates and durations) or by independent administration of separate formulations containing the active agents. “Co-administration” further includes concurrent administration (e.g. administration of CA4P and octreotide at the same time) and time varied administration (administration of CA4P at a time different from that of octreotide). The term “co-administration” is intended to convey that the multiple agents are being used together in a common regimen for the treatment of cancer.

Frequently, therapeutic regimens are administered in cycles. The methods described herein can consist of a single cycle or therapy or of a plurality of cycles. In the case, a plurality of cycles is utilized, the plurality can consist of a single regimen used repeatedly or cycles of different combinations and/or dosages used in succession. In some implementations, a treatment cycle comprises administration of CA4P 50-100 mg/m² of surface area once a week for four weeks, or alternatively for three weeks followed by a week of rest, in a 28-day cycle. In another implementation, consisting of a 21-day cycle, 50-100 mg/m²CA4P is administered on days 7, 14 and 21. These implementations can further comprise administering a single dose of a somatostatin analog, particularly an extended release formulation of such somatostatin analog, during each 21-day or 28-day cycle. In some implementations, octreotide additionally is administered on day 7 of a 21-day cycle or day 1 of a 28-day cycle.

The term “somatostatin analog” and any variants thereof, as used throughout the present application and claims, refer to proteinaceous material including single or multiple proteins or non-proteinaceous materials, and extends to those proteins having somatostatin or somatostatin-like activities, including the ability to bind to and/or otherwise modulate one or more somatostatin receptors SSTR1-SSTR5. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Examples of somatostatin analogs include somatostatin, lanreotide, octreotide and pasireotide, as well as formulations, both quick release and extended release, thereof.

The term “chemotherapeutic agent”, as used herein, refers to any chemical, for example a drug or compound, used or useful in the treatment of disease. For example, the term refers to cytostatic, cytotoxic, and/or anti-neoplastic drugs used to treat cancer or a combination of drugs used in a standardized cancer treatment regimen. Non-limiting examples of chemotherapeutic agents are alkylating agents (such as cisplatin, carboplatin, oxaliplatin, mechlorethamine, cyclophosphamide, chlorambucil, dacarbazine and streptozotocin), anti-metabolites (such as 5-FU, capecitabine, purine analogs azathioprine, mercaptopurine, or pyrimidine analogs), vinca alkaloids (such as vincristine, vinblastine, vinorelbine, vindesine), taxanes (for example paclitaxel, taxol, or docetaxel), podophyllotoxin and its derivatives (for example etoposide or teniposide), topoisomerase inhibitors (such as camptothecin, irinotecan or topotecan), amsacrine, epidophyllotoxin derivatives, anti-angiogenic agents (such as bevacizumab, cabozantinib, tivozanib, and thalidomide), tyrosine kinase inhibitors (such as sunitinib, sorafenib, erlotinib, gefitinib, axitinib and pazopanib), interferon alpha and antitumor antibiotics, such as dactinomycin, bleomycin, plicamycin, mitomycin.

One aspect of the invention is a pharmaceutical composition useful for treating carcinoid syndrome in a mammal, which composition comprises a vascular disrupting agent, such as CA4P or CA1dP, in combination with a pharmaceutically acceptable excipient. The composition is prepared in accordance with known formulation techniques to provide a composition suitable for oral, topical, transdermal, rectal, by inhalation, parenteral (intravenous, intramuscular, or intraperitoneal) administration, and the like. Detailed guidance for preparing compositions of the invention are found by reference to the 18^{th} or 19^{th} Edition of Remington's Pharmaceutical. Sciences, Published by the Mack Publishing Co., Easton, Pa. 18040. In certain implementations, the pharmaceutical composition further comprises a second therapeutic agent, such as a somatostatin analog or chemotherapeutic agent.

Unit doses or multiple dose forms are contemplated, each offering advantages in certain clinical settings. The unit dose would contain a predetermined quantity of active compound calculated to produce the desired effect(s) in the setting of treating carcinoid syndrome. The multiple dose form may be particularly useful when multiples of single doses, or fractional doses, are required to achieve the desired ends. Either of these dosing forms may have specifications that are dictated by or directly dependent upon the unique characteristic of the particular compound, the particular therapeutic effect to be achieved, and any limitations inherent in the art of preparing the particular compound for treatment of cancer.

A unit dose of vascular disrupting agent, particularly CA4P or CA1dP, will contain a therapeutically effective amount sufficient to treat carcinoid syndrome in a subject and may contain from about 1.0 to 1000 mg of compound, for example about 50 to 500 mg.

The vascular disrupting agent preferably is administered parenterally, e.g., intravenously, intramuscularly, intravenously, subcutaneously, or intraperitoneally. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of the compound may be prepared in suitable diluents such as water, ethanol, glycerol, liquid polyethylene glycol(s), various oils, and/or mixtures thereof, and others known to those skilled in the art.

The pharmaceutical forms suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thimerosal, or the like. It may also be preferable to include agents that alter the tonicity such as sugars or salts.

The vascular disrupting agent also can be administered orally in a suitable formulation as an ingestible tablet, a buccal tablet, capsule, caplet, elixir, suspension, syrup, trouche, wafer, lozenge, and the like. Generally, the most straightforward formulation is a tablet or capsule (individually or collectively designated as an “oral dosage unit”). Suitable formulations are prepared in accordance with a standard formulating techniques available that match the characteristics of the compound to the excipients available for formulating an appropriate composition. A tablet or capsule will preferably contain about 50 to about 500 mg of such a combretastatin compound.

The form may deliver a compound rapidly or may be a sustained-release preparation. The compound may be enclosed in a hard or soft capsule, may be compressed into tablets, or may be incorporated with beverages, food or otherwise into the diet. The percentage of the final composition and the preparations may, of course, be varied and may conveniently range between 1 and 90% of the weight of the final form, e.g., tablet. The amount in such therapeutically useful compositions is such that a suitable dosage will be obtained. Preferred compositions according to the current invention are prepared so that an oral dosage unit form contains between about 5.0 to about 50% by weight (% w) in dosage units weighing between 5 and 1000 mg.

The suitable formulation of an oral dosage unit may also contain: a binder, such as gum tragacanth, acacia, corn starch, gelatin; sweetening agents such as lactose or sucrose; disintegrating agents such as corn starch, alginic acid and the like; a lubricant such as magnesium stearate; or flavoring such a peppermint, oil of wintergreen or the like. Various other materials may be present as coating or to otherwise modify the physical form of the oral dosage unit. The oral dosage unit may be coated with shellac, a sugar or both. Syrup or elixir may contain the compound, sucrose as a sweetening agent, methyl and propylparabens as a preservative, a dye and flavoring. Any material utilized should be pharmaceutically-acceptable and substantially non-toxic. Details of the types of excipients useful may be found in the nineteenth edition of “Remington: The Science and Practice of Pharmacy,” Mack Printing Company, Easton, Pa. See particularly chapters 91-93 for a fuller discussion.

Another aspect of this invention is a method for treating carcinoid syndrome in a warm-blooded animal, which method comprises administering a therapeutically effective amount of a vascular disrupting agent. A vascular disrupting agent useful in this invention is administered to an appropriate subject in need of these agents in a therapeutically effective dose by a medically acceptable route of administration such as orally, parentally (e.g., intramuscularly, intravenously, subcutaneously, intraperitoneally), transdermally, rectally, by inhalation and the like.

With mammals, including humans, the effective amounts can be administered on the basis of body surface area. The interrelationship of dosages varies for animals of various sizes and species, and for humans (based on mg/m² of body surface) is described by E. J. Freireich et al., Cancer Chemother. Rep., 50(4):219 (1966). Body surface area may be approximately determined from the height and weight of an individual (see, e.g., Scientific Tables, Geigy Pharmaceuticals, Ardsley, N.Y. pp. 537-538 (1970)). A suitable dose range is from 1 to 1000 mg of equivalent per m² body surface area of a compound of the invention, for instance from 50 to 500 mg/m².

### V. EXAMPLES

### A. Example 1

### Tumor Necrosis in GH3 Rat Pituitary Tumors

Four female Wistar Furth rats, weighing approximately 160 g, were inoculated subcutaneously in the flanks with GH3 pituitary tumor cells. Tumors were typically used for experimentation when they reached approximately 1000-3000 mm³. Blood was taken from the tail vein of each rat 24 h before treatment with 50 mg/kg of vascular disrupting agent, ZD6126, formulated in 20% of 5% aqueous sodium bicarbonate in 80% phosphate buffered saline. The vascular disrupting agent was administered as a bolus injection through the tail vein. 24 h after treatment a further blood sample was taken from the tail vein and the rats euthanized. Tumors were excised, fixed in formalin and stained with H&E for histology. Necrosis was determined by image analysis (Image J). Blood samples were analyzed for prolactin and growth hormone at by ELISA using kits supplied by SPIbio.

Two rats showed the expected of necrosis 24 h after ZD6126 treatment (39 and 42% respectively) albeit at a lower level than previously obtained in a number of other studies. Two rats surprisingly showed little or no necrosis after a single dose of vascular disrupting agent.

Tumor-bearing rats displayed increased prolactin and growth hormone levels compared to control. In the rats in which tumor necrosis was apparent after VDA treatment, the levels of circulating hormones were significantly reduced. No reduction was seen in the animals in which necrosis was absent after treatment (Table 1; ±SD, A is the percent reduction). Since control animals had significant levels of both hormones (within the normal range) and since the VDA could not be expected to reduce normal host hormone production it might be reasonable to subtract a figure from each reading to allow for the persisting host-derived hormone. Although data on normal hormone levels were derived from a single control (non-tumor bearing) animal, the values are very much in agreement with those reported in the literature. Subtracting this value would give a rough estimate of the reduction in rats 1 and 2 of tumor-derived growth hormone of 72% and 40% and a reduction in tumor-derived prolactin of 65% and 33% respectively.

TABLE 1

| | | Growth Hormone | | | | Prolactin | | | |
|---|---|---|---|---|---|---|---|---|---|
| Rat | % necr. | pre | post | Δ | p | pre | post | Δ | p |
| 1 | 39 | 214 ± 11 | 91 ± 7 | 57 | 0.01 | 211 ± 28 | 90 ± 2 | 57 | 0.01 |
| 2 | 42 | 139 ± 8 | 102 ± 4 | 26 | 0.01 | 104 ± 3 | 78 ± 3 | 24 | 0.01 |
| 3 | 0 | 193 ± 4 | 204 ± 14 | −5 | NS | 134 ± 17 | 129 ± 5 | 4 | NS |
| 4 | 0 | 146 ± 4 | 171 ± 11 | −16 | 0.01 | 53 ± 3 | 52 ± 1 | 1 | NS |
| Tumor-free | n.d. | 46 ± 1 | n.d | n.d | n.d | 26 ± 1 | n.d | n.d | n.d |

### B. Example 2

### Hormone Production in Prolactinomas Treated with CA4P

Eighteen female Wistar Furth rats (Charles River, France), weighing approximately 130-140 g, were inoculated subcutaneously in the flanks with 1×10⁶ GH3 pituitary tumor cells. Tumors were typically used for experimentation when they reached approximately 1 cm in diameter, or 300-1000 mm³. Blood was taken from the tail vein of each rat 24 h before treatment with 100 mg/kg of vascular disrupting agent, CA4P, formulated in 20% of 5% aqueous sodium bicarbonate in 80% phosphate buffered saline. The CA4P was administered as a bolus injection through the tail vein. 24 h or 48 h after treatment a further blood sample was taken from the tail vein and the rats euthanized. Tumors were excised, fixed in formalin and stained with H&E for histology. Necrosis was determined by image analysis (Image J).

Prolactin and growth hormone were measured using commercial ELISA's (SPI/BIO Bertin Pharma/Cayman kits sourced from Bioquote Limited, York, UK). Tumor-derived prolactin levels were calculated assuming a historical basal level of 26 ng/ml. Treatment with CA4P resulted in a statistically lower amount of both total and tumor-derived prolactin (p<0.04, one-tailed T-test). Tumor-derived growth hormone levels were calculated by subtracting a historical value for background growth hormone of 46 ng/ml. Treatment with CA4P resulted in a statistically lower amount of both total and tumor-derived growth hormone (p<0.03, one-tailed t-test). All measurements for the control group were performed 24 hours after injection of saline.

TABLE 2

| | Prolactin (ng/ml ± SD) | | | Growth Hormone (ng/ml ± SD) | | | % tumor |
|---|---|---|---|---|---|---|---|
| | total | tumor-derived | n | total | tumor-derived | n | necrosis |
| Pretreatment | 87.0 ± 12.5 | 61.0 ± 12.5 | 4 | 88.5 ± 13.6 | 42.5 ± 13.6 | 14 | N/A |
| Control | 88.5 ± 8.3 | 62.5 ± 8.3 | 6 | 85.3 ± 10.7 | 39.3 ± 10.7 | 6 | 7.0 ± 9.1 |
| CA4P 24 h | 64.6 ± 19.1 | 38.6 ± 19.1 | 6 | 75.1 ± 13.6 | 29.1 ± 13.6 | 6 | 43.7 ± 29.6 |
| CA4P 48 h | 54.3 ± 20.2 | 28.3 ± 20.2 | 2 | 66.8 ± 7.8 | 20.8 ± 7.8 | 2 | 61.7 ± 27.4 |

Unlike the results found for ZD6126 (see Example 1), there does not appear to be a direct correlation between tumor necrosis and reduction of tumor-associated hormones. Table 3 summarizes the changes in tumor-derived growth hormone and the associated percent tumor necrosis for individual rats.

TABLE 3

| | treatment | tumor | tumor | GH % | % tumor |
|---|---|---|---|---|---|
| rat | group | GH pre- | GH post- | decrease | necrosis |
| R2.1 | control | 48.6 | 48.5 | 0.2 | 5 |
| R2.2 | control | 40.2 | 37.7 | 6.2 | 24 |
| R2.3 | control | 46.9 | 36 | 23.2 | 7 |
| R3.2 | control | 23.5 | 21.9 | 6.8 | 0 |
| R6.2 | control | 43.8 | 39.5 | 9.8 | 6 |
| R3.3 | CA4P 24 h | 42.9 | 15.8 | 63.2 | 83 |
| R4.1 | CA4P 24 h | 45.5 | 29.1 | 36.0 | 58 |
| R5.2 | CA4P 24 h | 61.7 | 48.4 | 21.6 | 3 |
| R5.3 | CA4P 24 h | 38.4 | 19.2 | 50.0 | 59 |
| R6.1 | CA4P 24 h | 45.2 | 42.4 | 6.2 | 59 |
| R4.3 | CA4P 48 h | 42.6 | 15.3 | 64.1 | 77 |
| R6.3 | CA4P 48 h | 40.6 | 26.3 | 35.2 | 30 |

### C. Example 3

### CA4P in the Treatment of Pancreatic Endocrine Tumors

Eight Pdx1-Cre:Men1floxed/floxed mice (described in Shen et al. Cancer Res. 2009, 69(5):1858-66) were divided into treated group (n=4) and control group (n=4). In an ongoing experiment, the mice from the treated group and control group were injected i.p. daily with CA4P (100 mg/kg) and saline, respectively, for three days monthly. The mice underwent a 24 h fast prior to collecting whole blood via a retro-orbital bleeding technique weekly at pre- and post-treatment. Glucose levels were monitored by enzymatic colorimetric assay. Insulin levels were monitored by enzyme-linked immunosorbent assay (ELISA) and are summarized in Table 4. All mice underwent a microPET/CT scan monthly. Anti-tumor activity was measured by standardized-uptake value (SUV) analysis. All mice were weighed three times per week as an indicator of toxicity.

TABLE 4

| Day | 0 | 4 | 11 |
|---|---|---|---|
| CA4P Serum Insulin | 3.695 ± 0.669 | 3.060 ± 1.037 | 2.495 ± 0.8722 |
| (μg/L) | | | |
| PBS Serum Insulin | 3.485 ± 0.66 | 3.645 ± 0.618 | 4.034 ± 0.726 |
| (μg/L) | | | |
| P (CA4P vs. PBS) | 0.6720 | 0.3699 | 0.0350 |

In a second experiment, six Pdx1-Cre:Men1floxed/floxed mice are divided into treated group (n=3) and control group (n=3). The mice from the treated group and control group are injected i.p. daily with CA4P (100 mg/kg) and saline, respectively, for three days. All mice from the second protocol are sacrificed on day 7. Tumor size and necrosis area is detected by H&E staining. Hypoxia in tumor cells and tumor-associated tissues is detected by PIMO. Vascular lumen area is detected by CD31 stating. Apoptosis is measured by TUNEL assay. Inhibition of tumor cells, tumor stem-like cells, endothelial cells and endothelial progenitor cells is measured by the combination of in vivo sequential labeling cell proliferation assay and immunofluorescent staining.

### D. Example 4

### Efficacy and Safety of CA4P in the Treatment of Carcinoid Syndrome

Twenty subjects with histologically confirmed diagnosis of carcinoid tumor or a carcinoid tumor of unknown location with liver metastasis and persistent clinical manifestations of carcinoid syndrome are enrolled in a single-arm open-label study. Upon entry to the study, each participant subjected to a variety of screening procedures including, but not limited to, pathology confirmation of carcinoid tumor, full physical examination, blood tests (chemistry, hematology, coagulation), baseline measurement of 5-HIAA plasma concentration, baseline measurement of 5-HIAA urine concentration, baseline measurement of chromogranin A plasma concentration, urinalysis and carcinoid syndrome symptom score. Typically a carcinoid score takes into consideration frequency of diarrhea, bowel movements per day, severity of flushing, and be a severity on a scale of 1-5. Patients with cardiac involvement at the time of screening are excluded.

Approximately 1 hour prior to CA4P administration, the subject receives 8 mg p.o. or i.v. dexamethasone and 500 mg p.o. acetaminophen/paracetamol. Subjects who have experienced hypertension requiring intervention on prior cycles, at the clinician's discretion, also receive amlodipine 5-10 mg p.o. or diltiazem 30-60 mg p.o. Plasma and urine concentrations of 5-HIAA and plasma concentration of chromogranin are measured.

Each subject receives 60 mg/m² combretastatin A-4 phosphate weekly on days 1, 8, 15 and 22 of a 28 day cycle. Subjects are treated for up to three cycles. Combretastatin A-4 phosphate is administered i.v. over 10 minutes. At the end of each cycle, plasma 5-HIAA, 24 hour urine for 5-HIAA, plasma chromogranin A, carcinoid syndrome score, concomitant medication and adverse event (if any) are recorded for each subject. One day 1 of each 28-day cycle, octreotide LAR is administered by intragluteal injection at a dose of 20-80 mg at the clinician's discretion.

At the conclusion of the study, approximately 30 days from last CA4P infusion, each participant subjected to a variety of screening procedures including, but not limited to, full physical examination, carcinoid syndrome score. Plasma 5-HIAA, 24 hour urine for 5-HIAA, plasma chromogranin A, blood tests (chemistry, hematology, and coagulation) and urinalysis.

The primary end points of this study are change in 5-HIAA and chromogranin from baseline and change in symptoms of carcinoid syndrome from baseline. The secondary end points are characterization of the safety of CA4P in combination with octreotide LAR and incidence of all adverse events.

### E. Example 5

### In Vivo Model of Establishment Carcinoid Syndrome

Carcinoid syndrome is established in mice, by injecting 1×10⁷ BON cells, a human carcinoid cell line (Jackson, et al. 2009 “Development and Characterization of a Novel In vivo Model of Carcinoid Syndrome” Clin Cancer Res 15(8):2747-2755), are injected intrasplenically into twenty male athymic nude mice (4-6 weeks; ˜25 g). For injection, the mice are anesthetized with isoflurane, a small left subcostal flank incision is made and the spleen exteriorized. Tumor cells (1×107 per 200 μl) are injected into the spleen with a 27-gauge needle. The spleen is returned to the abdomen, and the wound closed in one layer with wound clips.

Following intrasplenic injection of BON cells, mice are randomized into groups of five to receive treatment with vehicle (distilled H₂O, 200 μl, intraperitoneally, once every other day), octreotide (5 mg/kg, intraperitoneally, once every other day), combretastatin A-4 phosphate (50 mg/kg, intraperitoneally, once every other day), or combretastatin A-1 diphosphate (50 mg/kg, intraperitoneally, once every other day), with the first dose administered within 2 hours of splenic injection of the BON cells. The animals are treated weekly for 12 weeks. Occurrence of diarrhea is recorded.

Echocardiography is done at week 12, immediately before sacrifice. Spleen, heart and liver are collection for analysis and plasma is collected for 5-HT detection. On sacrifice, hearts, and segments of liver and spleen are immediately placed in 10% neutral buffered formalin for 24 h followed by 70% ethanol for 24 h.

After removal, to display cardiac valves, hearts are manually cut in a near-sagittal plane at 1 mm increments with emphasis on obtaining longitudinal sections of tricuspid and mitral valves. All tissue samples are paraffin-embedded, sections at 5 μm, and routinely deparaffinized and dehydrated. Sections of liver and spleen are stained with hematoxylin and eosin stain (H & E stain). Heart sections are stained with Movat pentachrome to display collagen, elastin and glycoaminoglycans characteristic of immature connective tissue. Less cardiac tissue fibrosis and valve impairment is indicative of effective treatment and/or prevention of carcinoid heart disease.

Liver samples are analyzed for number and extent of tumor metastasis. A decrease in the number of extent of liver metastases after treatment with combretastatin as compared to control is indicative of effective treatment of the underlying neuroendocrine tumor. 5-HT (serotonin) levels are measured vie enzyme immunoassay using commercial kits. Urinary 5-HIAA is measured by ELISA. Lower levels after treatment with combretastatin as compared to controls is indicative of effective treatment of carcinoid syndrome.

### F. Example 6

### In Vivo Model of Existing Carcinoid Syndrome

Carcinoid syndrome is established in mice as described above. Following intrasplenic injection of BON cells, mice are returned to their cages and monitored for 5 weeks. Mice then are randomized into groups of five to receive treatment with vehicle (distilled H₂O, 200 μl, intraperitoneally, once every other day), octreotide (5 mg/kg, intraperitoneally, once every other day), combretastatin A-4 phosphate (50 mg/kg, intraperitoneally, once every other day), or combretastatin A-1 diphosphate (50 mg/kg, intraperitoneally, once every other day. The animals are treated weekly for 12 weeks. Occurrence of diarrhea is recorded. Echocardiography, tissue analysis and biochemical assays are performed as described above.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1-19. (canceled)

20. A method of alleviating symptoms associated with increased hormone production by a neuroendocrine tumor comprising administering a therapeutically effective amount of a vascular disrupting to a mammal having a neuroendocrine tumor.

21. The method of claim 20, wherein the vascular disrupting agent is combretastatin A-4 phosphate, or a pharmaceutically acceptable salt thereof.

22. The method of claim 20, wherein the vascular disrupting agent is combretastatin A-1 diphosphate, or a pharmaceutically acceptable salt thereof.

23. The method of claim 20, further comprising administering a second therapeutic agent.

24. The method of claim 23, wherein the second therapeutic agent is a somatostatin analog.

25. The method of claim 24, wherein the somatostatin analog is octreotide.

26. The method of claim 20, wherein the neuroendocrine tumor is a carcinoid tumor, pancreatic neuroendocrine tumor, gastrinoma, insulinoma, VIPoma, glucagonoma or pituitary tumor.

27. The method of claim 26, wherein the neuroendocrine tumor is a carcinoid tumor.

28. The method of claim 20, wherein the hormone is selected from serotonin, chromogranin, neurotensin, vasoactive intestinal peptide, histamine, dopamine, kallikrein, substance P, insulin, prostaglandin, glucagon, gastrin, ACTH, somatostatin, and parathyroid hormone.

29. A method of treating carcinoid syndrome, the method comprising administering, to a mammal suffering from one or more symptoms of carcinoid syndrome, a therapeutically effective amount of a vascular disrupting agent.

30. The method of claim 29, wherein the vascular disrupting agent is a combretastatin.

31. The method of claim 30, wherein the combretastatin is combretastatin A-4, a combretastatin A-4 phosphate, or a pharmaceutically acceptable salt thereof.

32. The method of claim 30, wherein the combretastatin is combretastatin A-1, combretastatin A-1 diphosphate, or a pharmaceutically acceptable salt thereof.

33. The method of claim 29, further comprising administering a second therapeutic agent.

34. The method of claim 33, wherein the second therapeutic agent is a somatostatin analog.

35. The method of claim 34, wherein the somatostatin analog is octreotide.

36. The method of claim 29, wherein the symptoms of carcinoid syndrome are selected from the group consisting of a metabolic disorder, diarrhea, flushing, abdominal pain, heart disease, wheezing, bloating, and sweating.

37. The method of claim 29, wherein the symptoms of carcinoid syndrome include elevated hormone levels, wherein the hormone is selected from the group consisting of serotonin, chromogranin, neurotensin, vasoactive intestinal peptide, histamine, dopamine, kallikrein, substance P, insulin, prostaglandin, glucagon, gastrin, adrenocorticotropic hormone (ACTH), somatostatin, and parathyroid hormone.
